(19) United States
(12) Patent Application Publication
(10) Pub. No.: US 2020/0165210 A1
(43) Pub. Date: May 28, 2020

(54) METHOD FOR PREPARING AZOXYSTROBIN

(71) Applicant: SINON CORPORATION, TAICHUNG CITY (TW)

(72) Inventors: Chien-Hsing CHEN, TAICHUNG CITY (TW); Ming-Fang HSIEH, TAICHUNG CITY (TW); Chih-Da LIN, TAICHUNG CITY (TW); Chien-Yu LIU, TAICHUNG CITY (TW)

(21) Appl. No.: 16/241,446

(22) Filed: Jan. 7, 2019

(30) Foreign Application Priority Data
Nov. 28, 2018 (TW) 107142511

(51) Int. Cl. C07D 239/52; B01D 3/36; B01D 3/10

(52) U.S. Cl. CPC C07D 239/52; A01N 43/54; B01D 3/10; B01D 3/36

(57) ABSTRACT

A method for preparing azoxystrobin comprises the following steps: (a) mixing methyl (E)-2-[2-(6-chloropyrimidin-4-yloxy)phenyl]-3-methoxyacrylate, 2-cyanophenol, potassium carbonate, and 10-80 mol % of 1-methylpyrrolidine as a catalyst in an aprotic solvent to form a basic mixture, and reacting the basic mixture for 2-5 hrs at a temperature of 60-120° C.; and (b) subjecting the basic mixture after reaction in Step (a) to a first distillation under a reduced pressure of 80-120 torr at 70-80° C., to obtain azoxystrobin.

### BACKGROUND OF THE INVENTION

### 1. Field of the Invention

The present invention relates to a method for preparing a fungicidal agent, and particularly to a method for preparing azoxystrobin.

### 2. Description of the Related Art

The compound methyl (E)-2-[2-(6-(2-cyanophenoxy)pyrimidin-4-yloxy)phenyl]-3-methoxyacrylate is a widely used fungicidal agent in agriculture known as Azoxystrobin.

PCT Publication No. WO 9208703 discloses that the derivative of phenoxypyrimidine has fungicidal potency.

Moreover, U.S. Pat. No. 5,395,837 discloses a method for synthesizing a 4,6-bis(aryloxy)pyrimidine derivative, by reacting at 95-100° C. in N,N-dimethylformamide (DMF) as a solvent in the presence of potassium carbonate as a base and cuprous chloride (CuCl) as a catalyst. The yield is 64%, and the reaction scheme is as follows:

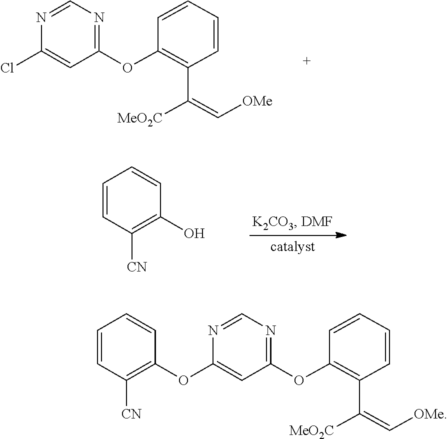 [SMILES: CO/C=C(/C(=O)OC)c1ccccc1Oc1cc(Cl)ncn1 ; N#Cc1ccccc1O ; CO/C=C(/C(=O)OC)c1ccccc1Oc1cc(Oc2ccccc2C#N)ncn1]

Furthermore, Bayer further discloses in PCT Publication No. WO 0172719 that a phenoxy derivative and a chloropyrimidine derivative are reacted at 50-80° C. in methyl isobutyl ketone as a solvent, in the presence of potassium carbonate as a base and 1,4-diazabicyclo[2,2,2]octane (DABCO) as a catalyst. The yield is 79-96%.

Then, Syngenta also discloses in EP Publication No. EP 1891020 that in the preparation of azoxystrobin, the yield is 86-98% when 0.1-40 mol % DABCO is added as a catalyst. The reaction scheme is as follows:

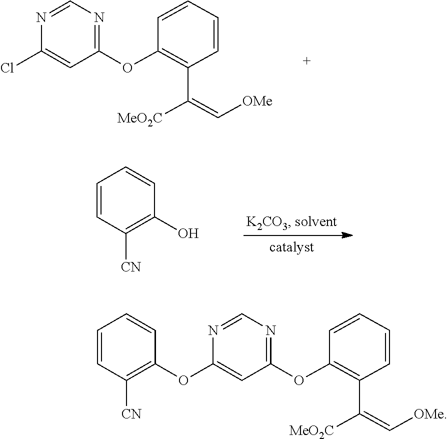 [SMILES: CO/C=C(/C(=O)OC)c1ccccc1Oc1cc(Cl)ncn1 ; N#Cc1ccccc1O ; CO/C=C(/C(=O)OC)c1ccccc1Oc1cc(Oc2ccccc2C#N)ncn1]

Therefore, as can be known from the above-mentioned prior art that although DABCO is previously often used as a catalyst, DABCO cannot be easily recycled and reused, and thus is not a suitable catalyst.

### SUMMARY OF THE INVENTION

A main object of the present invention is to provide a method for preparing azoxystrobin by using a catalyst that is easy to recycle and reuse in a process and is thus environmentally friendly. The present method is an environmentally friendly preparation method.

To achieve the above object, the present invention provides a method for preparing azoxystrobin, which comprises Step (a): mixing methyl (E)-2-[2-(6-chloropyrimidin-4-yloxy)phenyl]-3-methoxyacrylate, 2-cyanophenol, potassium carbonate, and 10-80 mol % of 1-methylpyrrolidine as a catalyst in an aprotic solvent to form a basic mixture, and reacting the basic mixture for 2-5 hrs at a temperature of 60-120° C.; and Step (b): subjecting the basic mixture after reaction in Step (a) to a first distillation under a reduced pressure of 80-120 torr at 70-80° C., to obtain azoxystrobin.

Therefore, in the method for preparing azoxystrobin provided in the present invention, 1-methylpyrrolidine is used as a catalyst that is easy to recycle and reuse in a process and is thus environmentally friendly. The present method is an environmentally friendly preparation method.

### DETAILED DESCRIPTION OF THE PREFERRED EMBODIMENTS

Step (a) comprises mixing methyl (E)-2-[2-(6-chloropyrimidin-4-yloxy)phenyl]-3-methoxyacrylate, 2-cyanophenol, potassium carbonate, and 10-80 mol % of 1-methylpyrrolidine as a catalyst in an aprotic solvent, to form a basic mixture; and reacting the basic mixture for 2-5 hrs at a temperature of 60-120° C., thus providing a preferred reaction environment for the basic mixture.

In Step (a), considering the effect and cost, the amount of the catalyst 1-methylpyrrolidine is preferably 10-80 mol %; preferably, the aprotic solvent is toluene, N,N-dimethylformamide, or methyl isobutyl ketone; and the basic mixture is firstly azeotropically distilled at 50-60° C. under 100-120 torr to remove water, whereby the yield of azoxystrobin is improved effectively. However, the removal of water by azeotropic distillation is not necessary.

Step (b) comprises subjecting the basic mixture after reaction in Step (a) to a first distillation under a reduced pressure of 80-120 torr at 70-80° C., to obtain azoxystrobin.

Step (c) comprises adding the product left after the first distillation under reduced pressure to the aprotic solvent and water, mixing, and standing for layer separation, collecting an upper organic layer, subjecting the organic layer to a second distillation under a reduced pressure of 20-40 torr at 50-60° C. to obtain a crude product, dissolving the crude product in methanol at 30-65° C., cooling to normal temperature and then to 0-5° C., filtering to obtain a crystalline solid, washing the crystalline solid with methanol, and then drying the crystalline solid at 40-65° C.

Step (c) is intended to enhance the purity of azoxystrobin. Where no high purity of azoxystrobin is required, Step (c) can be omitted. Therefore, Step (c) is not necessary in the present invention.

To explain the technical features of the present invention in detail, the present invention is described below with reference to preferred embodiments.

In a first preferred embodiment of the present invention, a method for preparing azoxystrobin is provided, which comprises mainly the following steps.

Methyl (E)-2-[2-(6-chloropyrimidin-4-yloxy)phenyl]-3-methoxyacrylate (97.6 gm), 2-cyanophenol (36.9 gm), and potassium carbonate (25.4 gm) are added to, mixed, and dissolved in an aprotic solvent (241 gm) at room temperature. In this embodiment, the aprotic solvent is, for example, toluene. Then, 1-methylpyrrolidine (10.2 gm, 0.4 eq) is added as a catalyst, and stirred until uniform to form a basic mixture. Subsequently, the basic mixture is heated to 80° C., and reacted for 3 hrs.

The basic mixture after reaction is subjected to a first distillation under a reduced pressure of 100 torr at 80° C., and the product left after the first distillation under reduced pressure is azoxystrobin. The distillate by the first distillation under reduced pressure is detected by gas chromatography (GC), and 1-methylpyrrolidine is determined to have a recovery rate of 96.3%.

Azoxystrobin left after the first distillation under reduced pressure is added to the aprotic solvent (241 gm) and water (114 gm), mixed, and stirred until azoxystrobin is completely dissolved. After standing for layer separation, an upper first organic layer is collected, and then a lower aqueous layer is collected and further added with the aprotic solvent (15 gm). Next, an upper second organic layer is collected. The first organic layer and the second organic layer are combined, and detected by high performance liquid chromatography (HPLC) to show a yield of azoxystrobin of 94.9%. The combined organic layer is subjected to a second distillation under reduced pressure at 50-60° C. under 20-40 torr, to remove the solvent. This gives a crude product. The crude product is dissolved in methanol (156 gm) at 60° C., cooled to normal temperature and then to 5° C., and filtered to obtain a crystalline solid. The crystalline solid was washed with methanol (13 gm) and then dried at 50° C., to obtain purified azoxystrobin (112.9 gm) as a solid (yield 91.5%).

The reaction scheme is as follows:

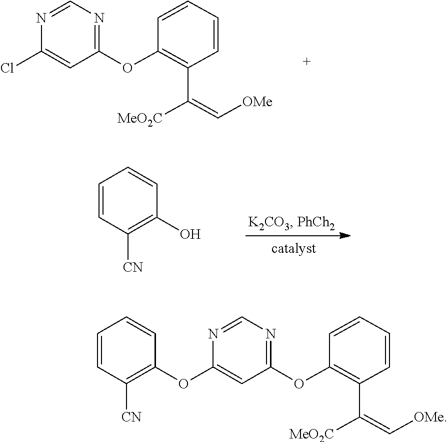 [SMILES: CO/C=C(/C(=O)OC)c1ccccc1Oc1cc(Cl)ncn1 ; N#Cc1ccccc1O ; CO/C=C(/C(=O)OC)c1ccccc1Oc1cc(Oc2ccccc2C#N)ncn1]

In a second preferred embodiment of the present invention, a method for preparing azoxystrobin is provided, which is substantially the same as that in the first preferred embodiment, except for the following differences.

The amount of the catalyst 1-methylpyrrolidine is (2.6 gm, 0.1 eq), the basic mixture is heated to 80° C., and the reaction time is 5 hrs.

The yield of azoxystrobin is 91.6%, and the recovery rate of the catalyst 1-methylpyrrolidine is 96.9%.

The yield of the resulting solid azoxystrobin (109.0 gm) is 88.3%.

In a third preferred embodiment of the present invention, a method for preparing azoxystrobin is provided, which is substantially the same as that in the first preferred embodiment, except for the following differences.

The amount of the catalyst 1-methylpyrrolidine is (5.1 gm, 0.2 eq), the basic mixture is heated to 80° C., and the reaction time is 5 hrs.

The yield of azoxystrobin is 91.9%, and the recovery rate of the catalyst 1-methylpyrrolidine is 97.1%.

The yield of the resulting solid azoxystrobin (109.4 gm) is 88.6%.

In a fourth preferred embodiment of the present invention, a method for preparing azoxystrobin is provided, which is substantially the same as that in the first preferred embodiment, except for the following differences.

The amount of the catalyst 1-methylpyrrolidine is (20.3 gm, 0.8 eq), the basic mixture is heated to 80° C., and the reaction time is 3 hrs.

The yield of azoxystrobin is 95.1%, and the recovery rate of the catalyst 1-methylpyrrolidine is 93.7%.

The yield of the resulting solid azoxystrobin (113.2 gm) is 91.7%.

In a fifth preferred embodiment of the present invention, a method for preparing azoxystrobin is provided, which is substantially the same as that in the first preferred embodiment, except for the following differences.

The basic mixture is heated to 120° C., and the reaction time is 2 hrs.

The yield of azoxystrobin is 93.1%, and the recovery rate of the catalyst 1-methylpyrrolidine is 87.2%.

The yield of the resulting solid azoxystrobin (110.8 gm) is 89.8%.

In a sixth preferred embodiment of the present invention, a method for preparing azoxystrobin is provided, which is substantially the same as that in the first preferred embodiment, except for the following differences.

The aprotic solvent is methyl isobutyl ketone (241 gm).

The yield of azoxystrobin is 95.4%, and the recovery rate of the catalyst 1-methylpyrrolidine is 95.9%.

The yield of the resulting solid azoxystrobin (113.6 gm) is 92.0%.

In a seventh preferred embodiment of the present invention, a method for preparing azoxystrobin is provided, which is substantially the same as that in the first preferred embodiment, except for the following differences.

The aprotic solvent is N,N-dimethylformamide (241 gm).

The yield of azoxystrobin is 92.8%, and the recovery rate of the catalyst 1-methylpyrrolidine is 96.8%.

The yield of the resulting solid azoxystrobin (110.5 gm) is 89.5%.

In an eighth preferred embodiment of the present invention, a method for preparing azoxystrobin is provided, which is substantially the same as that in the first preferred embodiment, except for the following differences.

The amount of the catalyst 1-methylpyrrolidine is (5.1 gm, 0.2 eq), triethylamine (6.1 gm, 0.2 eq) is added, the basic mixture is heated to 80° C., and the reaction time is 5 hrs.

The yield of azoxystrobin is 92.9%, and the recovery rate of the catalyst 1-methylpyrrolidine is 96.1%.

The yield of the resulting solid azoxystrobin (110.6 gm) is 89.6%.

In a ninth preferred embodiment of the present invention, a method for preparing azoxystrobin is provided, which is substantially the same as that in the first preferred embodiment, except for the following differences.

Removal of water by azeotropic distillation (at 60° C. under 100 torr) is performed, the catalyst 1-methylpyrrolidine is added, the basic mixture is heated to 80° C., and the reaction time is 4 hrs.

The yield of azoxystrobin is 97.3%, and the recovery rate of the catalyst 1-methylpyrrolidine is 96.4%. Therefore, the basic mixture is reacted in the absence of water to effectively increase the yield of azoxystrobin.

The yield of the resulting solid azoxystrobin (115.8 gm) is 93.8%.

In a tenth preferred embodiment of the present invention, a method for preparing azoxystrobin is provided, which is substantially the same as that in the first preferred embodiment, except for the following differences.

The catalyst 1-methylpyrrolidine recovered from the process is used, the amount of the catalyst 1-methylpyrrolidine is (10.2 gm, 0.4 eq), the basic mixture is heated to 80° C., and the reaction time is 3 hrs.

The yield of azoxystrobin is 94.1%, and the recovery rate of the catalyst 1-methylpyrrolidine is 95.5%. This indicates that the catalyst 1-methylpyrrolidine recovered from the process can be repeatedly used.

The yield of the resulting solid azoxystrobin (111.9 gm) is 90.7%.

It can be known from the results of the above preferred embodiments that in the method for preparing azoxystrobin provided in the present invention, 1-methylpyrrolidine is used as a catalyst that is easy to recycle and reuse in a process and is thus environmentally friendly. The present method is an environmentally friendly preparation method.

What is claimed is:

1. A method for preparing azoxystrobin, comprising the following steps:
(a) mixing methyl (E)-2-[2-(6-chloropyrimidin-4-yloxy)phenyl]-3-methoxyacrylate, 2-cyanophenol, potassium carbonate, and 10-80 mol % of 1-methylpyrrolidine as a catalyst in an aprotic solvent, to form a basic mixture; and reacting the basic mixture for 2-5 hrs at a temperature of 60-120° C.; and
(b) subjecting the basic mixture in Step (a) to a first distillation under a reduced pressure of 80-120 torr at 70-80° C., to obtain azoxystrobin.

2. The preparation method according to claim 1, further comprising the step of:
(c) adding the product left after the first distillation under reduced pressure to the aprotic solvent and water, mixing, and standing for layer separation, collecting an upper organic layer, subjecting the organic layer to a second distillation under a reduced pressure of 20-40 torr at 50-60° C. to obtain a crude product, dissolving the crude product in methanol at 30-65° C., cooling to normal temperature and then to 0-5° C., filtering to obtain a crystalline solid, washing the crystalline solid with methanol, and then drying the crystalline solid at 40-65° C.

3. The preparation method according to claim 1, wherein the aprotic solvent is toluene, N,N-dimethylformamide, or methyl isobutyl ketone.

4. The preparation method according to claim 1, wherein the basic mixture is reacted for 2-5 hrs at a temperature of 80-120° C.

5. The preparation method according to claim 1, wherein in Step (a), the basic mixture is reacted for 3 hrs at a temperature of 80° C.; and in Step (b), the first distillation under reduced pressure occurs at 80° C. under 100 torr.

6. The preparation method according to claim 1, wherein in Step (a), the basic mixture is reacted for 2 hrs at a temperature of 120° C.; and in Step (b), the first distillation under reduced pressure occurs at 80° C. under 100 torr.

7. The preparation method according to claim 1, wherein in Step (a), the basic mixture is firstly azeotropically distilled at 50-60° C. under 100-120 torr to remove water.
